(19) United States
(12) Patent Application Publication
(10) Pub. No.: US 2025/0033986 A1
(43) Pub. Date: Jan. 30, 2025

(54) EXTRUSION-ENCAPSULATED CALCIUM SALTS AS OPACIFYING/WHITENING AGENTS

(71) Applicant: CAPOL INC., St-Hubert (CA)

(72) Inventors: Plamen NIKOLOV, St-Hubert (CA); Pablo ELIZONDO, St-Hubert (CA)

(21) Appl. No.: 18/759,542

(22) Filed: Jun. 28, 2024

Related U.S. Application Data
(60) Provisional application No. 63/511,401, filed on Jun. 30, 2023.

(51) Int. Cl. C01F 11/18; A61K 8/11; A61K 8/19; A61Q 11/02

(52) U.S. Cl. CPC C01F 11/185; A61K 8/11; A61K 8/19; A61Q 11/02; C01P 2004/51; C01P 2004/61; C01P 2004/62; C01P 2006/12; C01P 2006/60; C01P 2006/62; C01P 2006/63; C01P 2006/64; C01P 2006/82

(57) ABSTRACT

An opacity modifying agent in dry particulate form as an alternative t titanium dioxide is described herein. The opacity modifying agent may comprise a calcium salt encapsulated in a water-insoluble protein matrix via extrusion encapsulation, leading to the encapsulated calcium salt having higher opacity than that of the unencapsulated calcium salt. In embodiments, the opacity modifying agent may comprise calcium carbonate encapsulated in an alkaline rice protein extract having a whiteness approaching that of calcium carbonate. Processes for producing the opacity modifying agents, as well as their uses in food, cosmetic, or personal care products are also described herein.

### CROSS-REFERENCE TO RELATED APPLICATION(S)

This application claims the benefit of U.S. provisional application No. 63/511,401 filed on Jun. 30, 2023, which is incorporated herein by reference in its entirety.

### BACKGROUND

The present description relates to opacifying and/or whitening agents suitable for use in the food, cosmetic, and personal care industries, as alternatives to titanium dioxide. More specifically, the present description relates opacifying and/or whitening agents based on calcium salts encapsulated in a water-insoluble protein matrix via extrusion encapsulation.

Titanium dioxide (TiO₂) is a water insoluble inorganic compound that has been widely used as a white pigment and opacifier. For many years, TiO₂ has been used as an agent for coloring or whitening in a variety of foods, including baked goods, candies and other confectionery goods, dairy products, etc. It is also used in pet foods. Other known applications for TiO₂ include the fields of pharmaceutical products, cosmetics, paints, and more. TiO₂ is known to be well-suited for the above-mentioned applications due to its chemical structure allowing for good light refraction and due to particle size, which allows for even dispersion in a formulation.

In early 2022, the European Union banned the use of TiO₂ as a food additive due to the identification of potential carcinogenic effects, with the possibility of other jurisdictions following suit in light of growing consumer pressure. In light of this, there have been significant efforts to develop safer alternatives to TiO₂ for use as opacifiers and/or whitening agents, particularly in the food, cosmetic, and personal care industries. However, significant challenges exist in this regard, with a report from the U.S. Department of Agriculture (USDA) reciting that “there are no good alternatives to titanium dioxide that can provide similar pigment/opacity properties” and that “indicated replacements require studies and regulatory filings which would take significant time, up to 10 years or more and reformulation cost estimates range from $600,000 to $1.8 million per product depending on the complexity of the product, which would be passed on to the consumer” (de Belder, 2022).

Alternatives to TiO₂ that have been considered include starched-based products such as corn and rice starches, as well as rice flours, alone or in combination with other substances. Starches, however, tend to retain moisture and large quantities may be required when compared to TiO₂.

Therefore, there is a need for the development of new and safer opacifiers and/or whitening agents as alternatives to TiO₂.

### SUMMARY

In a first aspect, described herein is an opacity modifying agent in dry particulate form comprising or consisting essentially of a calcium salt encapsulated in a water-insoluble protein matrix via extrusion encapsulation, wherein the opacity of the encapsulated calcium salt is greater than that of the unencapsulated calcium salt.

In a further aspect, described herein is a method for increasing the opacity of a calcium salt, the method comprising encapsulating the calcium salt in a water-insoluble protein matrix via extrusion encapsulation.

In a further aspect, described herein is a process for producing an opacity modifying agent in dry particulate form. The process generally comprises: (i) providing a calcium salt to be encapsulated and a water-insoluble protein-rich mixture; (ii) mixing the calcium salt, the water-insoluble protein-rich mixture, and water in an extruder at high shearing and pressure to raise the temperature, thereby enabling the formation of a melt; (iii) cooling the melt and extruding at temperatures comprising 45-60° C. to facilitate melt formation and/or encapsulation of the calcium salt; and (iv) drying and pulverising or grinding the extruded mixture into a dry particulate form.

In a further aspect, the opacity modifying agent described herein may be for use as an additive in a food product (e.g., a confectionary), a cosmetic product, or in a personal care product.

In a further aspect, described herein is a food, cosmetic, or personal care product comprising the opacity modifying agent as described herein.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows opacity measurements of the rice protein-encapsulated CaCO₃ agents described herein (with 30 wt % or 40 wt % CaCO₃ concentrations), compared to unencapsulated calcium carbonate, normalized for CaCO₃ content.

FIGS. 2A and 2B compare the priming ability of rice protein-encapsulated calcium carbonate with that of titanium dioxide in hard sugar panning applications. Chocolate lentils were engrossed with 30 layers of a 70 brix sugar solution containing either a 0.25% solution of titanium dioxide (FIG. 2A) or a 1.0% solution of the 30 wt % extrusion-encapsulated CaCO₃ solution prepared as described in Example 3 (FIG. 2B).

### DETAILED DESCRIPTION

Described herein are compositions and methods relating to the making and production of opacifying and/or whitening agents suitable for use as alternatives to titanium dioxide. In some aspects, the present subject-matter stems from the demonstration herein that calcium salts encapsulated in a protein-rich matrix via extrusion encapsulation possess improved opacifying and whitening properties. In some embodiments, described herein is a method for improving the opacity of calcium salt particles, such as calcium carbonate, the method comprising subjecting the calcium salt particles to extrusion encapsulation in an alkaline rice protein-rich matrix. Advantageously, the composition or agent described herein has superior or enhanced opacity/whitening power when compared to regular, food additive-grade calcium salts such as calcium carbonate, thereby providing an agent, or a composition, with superior or improved opacifying and/or whitening properties.

In a first aspect, described herein is an opacity modifying agent (or opacifier) in dry particulate form comprising or consisting essentially of a calcium salt encapsulated in a water-insoluble protein matrix via extrusion encapsulation. In some embodiments, the opacity of the encapsulated calcium salt is greater than that of the unencapsulated calcium salt and/or the encapsulated calcium salt has a greater resistance to acidic environments as compared to the unencapsulated calcium salt. As used herein, the expression “consisting essentially of” in the context of opacity modifying agents refers to the principal ingredients in the opacity modifying agents that provide the main functionality and utility of the agents as an opacifiers and/or whitening agents, but does not exclude the inclusion of other components (e.g., additives, stabilizers, preservatives) in lesser relative quantities so long as the other components do not abrogate the functionality imparted by the principal ingredients.

In some embodiments, the calcium salt described herein may comprise or consist of: calcium carbonate, calcium chloride, calcium pantothenate, calcium silicate, calcium lactobionate, calcium phosphate, monobasic calcium phosphate, calcium ascorbate, calcium sorbate, calcium acetate or diacetate, calcium hexametaphosphate, calcium pyrophosphate, calcium alginate, calcium citrate, calcium gluconate, calcium glycerophosphate, calcium hydroxide, calcium iodate, calcium lactate, calcium oxide, calcium propionate, calcium stearate, calcium sulfate, calcium disodium (or calcium disodium ethylene-diaminetetraacetate (EDTA)), and/or other calcium salts or calcium salts derivatives. In some embodiments, the calcium salt described herein may be a calcium salt approved for use as a food additive and/or coloring agent by a regulatory body (e.g., in the United States, in Europe, in Canada, or elsewhere).

In a preferred embodiment, the calcium salt described herein comprises or consists of calcium carbonate. Calcium carbonate is registered for use as a food additive under European Directive 1129/2011 (E170). It is also permitted as a food additive in the US and used for coloring drugs and food as well as a source of dietary calcium and as a firming agent. Calcium carbonate is a substance, generally a white powder, that is found in nature or can be prepared from other compounds such as calcium oxide or calcium chloride. Food-grade calcium carbonate is usually produced from naturally occurring limestone by grinding or from other mineral sources or from eggshells or seashells. It can also be synthetically prepared by precipitation.

In some embodiments, the water-insoluble protein matrix described herein may comprise or consist of a water-insoluble plant protein matrix. In some embodiments, the water-insoluble protein matrix described herein may comprise or consist of a water-insoluble rice protein extract, a water-insoluble soy protein extract, a water-insoluble pea protein extract, a water-insoluble sunflower protein extract, a water-insoluble oat protein extract, a water-insoluble fibrous plant protein extract, or any combination thereof. In some embodiments, the water-insoluble protein matrix may comprise or consist of a water-insoluble alkaline protein extract. In some embodiments, the water-insoluble protein matrix is produced from a protein extract raw material having (or selected based on having) a whiteness similar to that of the unencapsulated calcium salt. In some embodiments, the water-insoluble protein matrix is produced from a protein extract raw material having (or selected based on having) a whiteness defined by the following CIELAB and CIELCh coordinates: L* value from 84 to 100; a* value from 0 to 2; b* value from 0 to 16; C* value from 0 to 16; and h° value from 80 to 87. In some embodiments, the water-insoluble protein matrix is or is from a protein extract having an amino acid profile characterized by the conversion of cysteine to its oxidized cystine form. The amino acid profile of a protein extract is indicative and characteristic of the extraction process used to obtain the protein extract, which is demonstrated herein to impact the opacity and/or whiteness of the opacity modifying agent described herein.

In preferred embodiments, the water-insoluble protein matrix described herein may comprise or consist of a water-insoluble rice protein matrix. Rice protein, or alkaline rice protein, is an isolate from rice and is an alternative to soy protein in the food industry due to its decreased allergy potential, compared to dairy, egg or soy protein. Rice protein extracts (including isolates or concentrates) can be generated by treating rice with carbohydrate-breaking enzymes that remove the starch from the proteins. Rice protein extracts can thus be considered a by-product of rice starch production and have been given little consideration so far as a whitening/opacifying agent. Rice protein is also advantageous because of its insolubility and general light coloration during processing. However, despite this general light coloration, rice protein from different sources or suppliers may be of uneven, or different, baseline whiteness, opacity or coloration. Rice protein from different sources or suppliers can also have different amino acid compositions. Thus, in some embodiments, the rice protein extracts described herein may have the color and/or amino acid profile described above.

In some embodiments, the opacity modifying agent described herein may comprise about 20-50%, 25-45%, or 30-40% of the calcium salt by weight. In some embodiments, the opacity modifying agent described herein may comprise about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% of the calcium salt by weight. In some embodiments, the opacity modifying agent described herein may comprise about 50-80%, 55-80%, 55-75%, or about 60-70% of the water-insoluble protein matrix by weight. In some embodiments, the opacity modifying agent described herein may comprise about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80% of the water-insoluble protein matrix by weight. In some embodiments, the opacity modifying agent described herein may comprise greater than 20, 21, 22, 23, 24, 25, 26, 27, 28, 29, or 30% by weight of the calcium salt, and up to 70, 71, 72, 73, 74, 75, 76, 77, 78, 79, or 80% by weight of the water-insoluble protein matrix. In some embodiments, the opacity modifying agent described herein may comprise about 30% to 45% by weight of the calcium salt, and about 55% to 70% by weight of the water-insoluble protein matrix. In some embodiments, the opacity modifying agent described herein may comprise calcium carbonate as the calcium salt present at 30-40% by weight, and may comprise an alkaline rice protein extract as the water-insoluble protein matrix present at 60-70% by weight. For further clarity, the above-mentioned percentages refer to the opacity modifying agent fully dried.

In some embodiments, the opacity modifying agent described herein may have an average particle size of about 10 to 30 microns. In some embodiments, the opacity modifying agent in dry particulate form described herein may have an average particle size of about 5 to 50 microns. In some embodiments, the opacity modifying agent in dry particulate form described herein may have an average particle size of about 5 to 100 microns.

In some embodiments, the opacity modifying agent described herein may have a color profile similar to that of the calcium salt in unencapsulated form. In some embodiments, the opacity modifying agent described herein may have a whiteness defined by the following CIELAB and CIELCh coordinates: L* value from 84 to 100; a* value from 0 to 2; b* value from 0 to 16; C* value from 0 to 16; and h° value from 80 to 87.

In some embodiments, various additives or additional agents can be added to the encapsulated calcium salt to fine-tune or improve further its white color property, to decrease or reduce an eventual beige coloration and/or to increase opacity further.

In some embodiments, the opacity modifying agent described herein may be for use as an additive in a food product (e.g., a confectionary), a cosmetic product, or in a personal care product. In some aspects, described herein is a food, cosmetic, or personal care product comprising the opacity modifying agent as described herein or produced by a process as described herein.

In some aspects, described herein is a method for increasing the opacity of a calcium salt, the method comprising encapsulating the calcium salt in a water-insoluble protein matrix via extrusion encapsulation. In some embodiments, the method produces an opacity modifying agent as described herein.

In some aspects, described herein is a process for producing an opacity modifying agent in dry particulate form. In some embodiments, the process comprises: (i) providing a calcium salt to be encapsulated and a water-insoluble protein-rich mixture; (ii) mixing the calcium salt, the water-insoluble protein-rich mixture, and water in an extruder at high shearing and pressure to raise the temperature, thereby enabling the formation of a melt; (iii) cooling the melt and extruding at temperatures comprising 45-60° C. to facilitate melt formation and/or encapsulation of the calcium salt; and (iv) drying and pulverising or grinding the extruded mixture into a dry particulate form. In some embodiments, the extruder may be operated at a screw speed of about 900 rpm, at a temperature of about 55° C., and/or at a (air) humidity of between about 30% to about 38%. In some embodiments, the dried extruded mixture may be pulverized or ground to an average particle size of about 10 to 30 microns. In some embodiments, the dried extruded mixture may be pulverized or ground to an average particle size of about 5 to 50 microns, or about 5 to 100 microns.

In some embodiments, the process described herein may comprise premixing a calcium salt powder and a protein powder, for example using a blender, such as a powder blender. The premix of calcium salt powder and protein powder may then be fed into an extruder. In some embodiments, the calcium salt powder and protein powder may also be fed into the extruder separately, so that the mixing takes place into the extruder. In some embodiments, water may be added to the mix of calcium salt powder and protein powder when the mix is fed into the extruder or after the mix is fed into the extruder. In some embodiments, the mix fed into the extruder may be composed of about 60-90% powder and about 10-40% water. In some embodiments, the mix fed into the extruder may be composed of about 60% powder and about 40% water composition.

In some embodiments, after introduction of the mix into the extruder, the extruder sections may be set at about 30-70° C. and 800-950 rpm screw speed. These parameters may advantageously minimize residence time and allow higher throughput. In another embodiment, the extruder sections may also be set at 55° C. and 900 rpm screw speed.

### EXAMPLES

### Example 1: Comparison of Opacities of Titanium Dioxide and Calcium Carbonate

Calcium salts, such as calcium carbonate (CaCO₃), have been explored as potential replacements for titanium dioxide in terms of whitening agents, but their inability to approach the opacity of titanium dioxide represents a significant drawback. The opacities of titanium dioxide and calcium carbonate were measured by dispersing the powders at 10% in a hydroxypropyl methylcellulose (HPMC) solution followed by a drawdown onto a sheet that is half white and half black. The sheet was then allowed to dry completely after which the color difference between the respective white and black backgrounds of the sheet was quantified using a colorimeter. Representative results of an experiment performed in triplicate using calcium carbonate from two different suppliers are shown in Table 1.

TABLE 1
Comparison of opacities of TiO₂ and CaCO₃

| | Opacity | St. Dev. |
|---|---|---|
| TiO₂ | 73.7 | 9.4 |
| CaCO₃ (supplier A) | 5.6 | 1.1 |
| CaCO₃ (supplier B) | 11.6 | 3.9 |

Although some variability was observed between the opacities of different calcium carbonate suppliers (e.g., suppliers A vs. B), the results in Table 1 show that, at equal amounts, the opacity of calcium carbonate was found to be 6- to 13-fold lower than that of titanium dioxide. Alternatively stated, several fold higher amounts of calcium carbonate would need to be employed in order to achieve comparable opacity to titanium dioxide, which may not be desirable or feasible in the majority of applications.

A further drawback of calcium salts such as calcium carbonate relative to titanium dioxide is the former's sensitivity to acidic environments, resulting in fizzing or gas release, which limits their commercial applications. Thus, methods of increasing the opacity of calcium salts such as carbonate while increasing their resistance to acidic environments were explored.

### Example 2: Extrusion Encapsulation of Calcium Carbonate in Rice Protein Extract Increases Acid Resistance and Opacity

We have previously employed melt extrusion to encapsulate labile coloring agents in a water-insoluble protein-rich matrix, as described for example in patent nos. U.S. Pat. Nos. 9,687,807 and 10,981,136. As the main objective of those processes was to produce vibrant water-insoluble coloring agents, raw materials and extrusion parameters were selected and employed in order to encapsulate the coloring agents in a protein-rich matrix that enables the proper expression of their colors. Briefly, the ingredients (i.e., labile coloring agent, protein-rich mixture, and water) were mixed in an extruder to form a melt, and then the melt was extruded with active cooling at temperature settings that did not exceed 35° C. in order to minimize browning of the water-insoluble protein by the Maillard reaction. The extruded mixture was then dried and milled/ground to form a dry particle coloring agent.

We performed early experiments attempting to encapsulate calcium carbonate using the same extrusion conditions and rice protein concentrate (Remypro™ N80+) as described in U.S. Pat. Nos. 9,687,807 and 10,981,136, but these resulted in relatively low encapsulation efficiencies. Encapsulation was improved by raising the upper threshold extruder temperature settings to about 55° C. and increasing the screw speed to about 900 rpm, but these modifications resulted in an undesirable yellowing of the encapsulated calcium carbonate. Interestingly, subsequent acid exposure tests on the encapsulated calcium carbonate revealed not only an increased resistance to acidic pH (as measured by CO₂ production upon immersion in a weak acid), but also resulted in an increase in opacity as compared to unencapsulated calcium carbonate (data not shown).

Different options were explored to reduce the yellowing of the encapsulated calcium carbonate, without diminishing encapsulation efficiency. Among the options explored was the testing and screening of different sources and preparations of water-insoluble protein. Initially, enzymatic rice protein extracts were favored over alkaline protein extracts, as it is conventionally thought that the former may be less susceptible to browning via the Maillard reaction, which is known to be promoted by alkaline conditions. Surprisingly, it was found that alkaline rice protein extracts resulted in less yellowing of the rice protein-encapsulated calcium carbonate. Furthermore, amongst the rice protein extracts tested in extrusion encapsulations of calcium carbonate, it was found rice protein extracts having a color profile more similar to that of calcium carbonate prior to extrusion, yielded optimal results in terms of both opacity and maintaining the original whiteness of the encapsulate post-extrusion. Examples of color profiles of some of the raw materials discussed herein are shown in Table 2 below, using coordinates of the CIELAB-based CIELCh color space. It was also observed that alkaline rice protein extracts having color profiles more similar to that of calcium carbonate also had amino acid compositions distinct from the more yellow rice protein sources, such as the conversion of nearly all cysteine residues to their oxidized cystine form.

TABLE 2
Color analysis of raw materials (CIELCh color space)

| Raw material | L* | a* | b* | C* | h° |
|---|---|---|---|---|---|
| Rice protein concentrate | 79.25 | 2.87 | 20.8 | 21 | 82.15 |
| (Remypro ™ N80+) | | | | | |
| Alkaline rice protein extract (used in | 87.93 | 1.15 | 11.51 | 11.57 | 84.3 |
| Example 3) | | | | | |
| CaCO₃ (supplier A) | 97.15 | 0.28 | 1.68 | 1.68 | 80.85 |
| CaCO₃ (supplier B) | 96.23 | 0.13 | 2.4 | 2.4 | 86.92 |

### Example 3: Preparation of Extrusion-Encapsulated Calcium Carbonate Opacifying/Whitening Agent

Calcium carbonate powder and the alkaline rice protein extract powder shown in Table 2 were premixed in a blender and then fed into an extruder, along with water, at a ratio of approximately 62% powder and 38% water composition by mass. Extruder screw speed was set to about 900 rpm. Initial extruder sections were set to temperatures of about 35° C., middle sections were set to about 55° C., and final sections were set to about 5° C. The extrudate exited the extruder as pellets at a temperature of approximately 40° C. and a humidity range of approximately 30 to 38%. The extrudate pellets were of a size of approximately one (1) centimeter per one centimeter. The pellets were then directly run through an airswept pulverizer, which dried the pellets down to approximately 10% humidity and broke the pellets down to a rough powder with particles of a size of approximately 150 to 300 micrometers. This rough powder was then grinded through a jet mill in order to obtain a final percentage humidity below 7% and a particle size between approximately 10 to 30 microns. The final concentration of the rice protein-encapsulated CaCO₃ agent was 67% rice protein, 28% calcium carbonate, and 5% water. When fully dried, the encapsulated CaCO₃ agent was composed of 70% rice protein and 30% calcium carbonate by weight. In some production lots, the amount of alkaline rice protein extract powder was increased, resulting in an encapsulated CaCO₃ agent composed of 60% rice protein and 40% calcium carbonate by weight, when fully dried.

### Example 4: Measurement of Opacity of Extrusion-Encapsulated Calcium Carbonate Opacifying/Whitening Agent

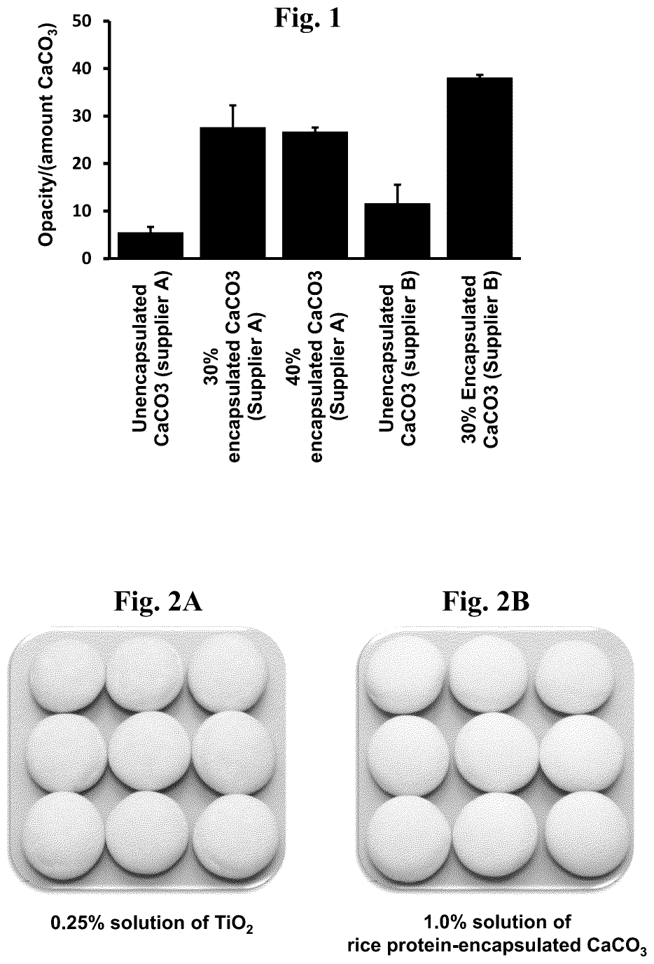

The extrusion-encapsulated calcium carbonate opacifying/whitening agents produced as described in Example 3 were measured for opacity as described in Example 1, and compared to the opacity of unencapsulated calcium carbonate. Representative results of an experiment performed in triplicate using calcium carbonate from two different suppliers (A and B) are shown in Table 2 and in FIG. 1, with the opacity measurements being normalized to the amount of calcium carbonate in each product.

TABLE 3
Comparison of opacities of encapsulated
and unencapsulated CaCO₃

| | | Opacity/ | St. |
|---|---|---|---|
| | | (amount CaCO₃) | Dev. |
| CaCO₃ | Unencapsulated CaCO₃ | 5.6 | 1.1 |
| Supplier A | 30 wt % encapsulated CaCO₃ | 27.7 | 4.6 |
| | 40 wt % encapsulated CaCO₃ | 26.7 | 0.9 |
| CaCO₃ | Unencapsulated CaCO₃ | 11.6 | 3.9 |
| Supplier B | 30 wt % encapsulated CaCO₃ | 38.1 | 0.5 |

The results shown in Table 3 and FIG. 1 show that rice protein-encapsulated calcium carbonate exhibited several fold higher opacity than the unencapsulated calcium carbonate controls.

### Example 5: Measurement Acid-Resistance of Extrusion-Encapsulated Calcium Carbonate Opacifying/Whitening Agent

The acid resistance of the extrusion-encapsulated calcium carbonate opacifying/whitening agents produced as described in Example 3, in comparison to unencapsulated calcium carbonate controls, were measured by immersion in a weak acid solution (0.1 M HCl) for a fixed period of time and then measuring the production of carbon dioxide gas released (CO₂; in mL) collected in an inverted graduated cylinder. The volume of carbon dioxide gas released was then normalized to the amount of calcium carbonate in each sample. Representative results measuring the average volume of carbon dioxide released across multiple different production lots are shown in Table 4.

TABLE 4
Comparison of CO₂ release between encapsulated
and unencapsulated CaCO₃

| | | Average volume CO₂ |
|---|---|---|
| | | released (mL) ± SEM |
| CaCO₃ | Unencapsulated CaCO₃ | 45.1 ± 2.0 |
| Supplier A | 30 wt % encapsulated CaCO₃ | 29.1 ± 2.7 |
| | 40 wt % encapsulated CaCO₃ | 26.5 ± 2.7 |

The results in Table 4 shows that rice protein-encapsulated calcium carbonate exhibited a 35-40% average decrease in the volume of carbon dioxide gas released, demonstrating an improved resistance to acidic environments.

### Example 6: Use of Extrusion-Encapsulated Calcium Carbonate Opacifying/Whitening Agent as an Opacifying Primer in Hard Sugar Panning

In hard sugar panning, a white or pale base coat (or primer) must be applied to cover a dark surface (e.g., a chocolate lentil) and achieve optimal opacity/whitening before subsequent coating with various coloring agents. FIGS. 2A and 2B compare the priming ability of rice protein-encapsulated calcium carbonate with that of titanium dioxide in hard sugar panning applications. Chocolate lentils were engrossed with 30 layers of a 70 brix sugar solution containing either a 0.25% solution of titanium dioxide (FIG. 2A) or a 1.0% solution of the 30 wt % extrusion-encapsulated CaCO₃ solution (FIG. 2B) prepared as described in Example 3. The results demonstrate that extrusion-encapsulated calcium carbonate possesses sufficient opacity and whiteness to provide a viable alternative to titanium dioxide.

### REFERENCES

De Belder T. (2022). Titanium Dioxide Banned as a Food Additive in the EU. Report from the U.S. Department of Agriculture.
U.S. Pat. No. 9,687,807
U.S. Pat. No. 10,981,136

1. An opacity modifying agent in dry particulate form comprising or consisting essentially of a calcium salt encapsulated in a water-insoluble protein matrix via extrusion encapsulation, wherein the opacity of the encapsulated calcium salt is greater than that of the unencapsulated calcium salt.

2. The opacity modifying agent of claim 1, wherein the calcium salt comprises or consists of: calcium carbonate, calcium chloride, calcium pantothenate, calcium silicate, calcium lactobionate, calcium phosphate, monobasic calcium phosphate, calcium ascorbate, calcium sorbate, calcium acetate or diacetate, calcium hexametaphosphate, calcium pyrophosphate, calcium alginate, calcium citrate, calcium gluconate, calcium glycerophosphate, calcium hydroxide, calcium iodate, calcium lactate, calcium oxide, calcium propionate, calcium stearate, calcium sulfate, calcium disodium (or calcium disodium ethylene-diaminetetraacetate (EDTA)), and/or other calcium salts or calcium salts derivatives.

3. The opacity modifying agent of claim 1, wherein the calcium salt comprises or consists of calcium carbonate.

4. The opacity modifying agent of claim 1, wherein the water-insoluble protein matrix comprises or consists of a water-insoluble plant protein matrix.

5. The opacity modifying agent of claim 1, wherein the water-insoluble protein matrix comprises or consists of a water-insoluble alkaline protein extract.

6. The opacity modifying agent of claim 1, wherein the water-insoluble protein matrix comprises or consists of a water-insoluble rice protein extract.

7. The opacity modifying agent of claim 1, wherein the water-insoluble protein matrix comprises or consists of a water-insoluble soy protein extract or a water-insoluble pea protein extract.

8. The opacity modifying agent of claim 1, wherein the water-insoluble protein matrix is produced from a protein extract raw material having a whiteness similar to that of the unencapsulated calcium salt.

9. The opacity modifying agent of claim 1, wherein the water-insoluble protein matrix and/or the opacity modifying agent has whiteness defined by the CIELAB and CIELCh coordinates: L* value from 84 to 100; a* value from 0 to 2; b* value from 0 to 16; C* value from 0 to 16; and h° value from 80 to 87.

10. The opacity modifying agent of claim 1, wherein the water-insoluble protein matrix is or is from a protein extract characterized by the conversion of cysteine to its oxidized cystine form.

11. The opacity modifying agent of claim 1, comprising about 20-50% by weight of the calcium salt, and about 50-80% by weight of the water-insoluble protein matrix.

12. The opacity modifying agent of claim 1, comprising greater than 20, 21, 22, 23, 24, 25, 26, 27, 28, 29 or 30% by weight of the calcium salt, and up to 70, 71, 72, 73, 74, 75, 76, 77, 78, 79, or 80% by weight of the water-insoluble protein matrix.

13. The opacity modifying agent of claim 1, comprising about 30% to about 45% by weight of the calcium salt, and about 55% to about 70% by weight of the water-insoluble protein matrix.

14. The opacity modifying agent of claim 1, wherein the calcium salt is calcium carbonate present at 30-40% by weight, and the water-insoluble protein matrix is an alkaline rice protein extract present at 60-70% by weight.

15. The opacity modifying agent of claim 1, which has an average particle size of about 10 to about 30 microns.

16. (canceled)

17. (canceled)

18. A method for increasing the opacity of a calcium salt, the method comprising encapsulating the calcium salt in a water-insoluble protein matrix via extrusion encapsulation.

19. The method of claim 18, wherein the method produces an opacity modifying agent comprising about 20-50% by weight of the calcium salt, and about 50-80% by weight of the water-insoluble protein matrix, and having a whiteness defined by the CIELAB and CIELCh coordinates: L* value from 84 to 100; a* value from 0 to 2; b* value from 0 to 16; C* value from 0 to 16; and h° value from 80 to 87.

20. A process for producing an opacity modifying agent in dry particulate form, the process comprising:
(i) providing a calcium salt to be encapsulated and a water-insoluble protein-rich mixture;
(ii) mixing the calcium salt, the water-insoluble protein-rich mixture, and water in an extruder at high shearing and pressure to raise the temperature, thereby enabling the formation of a melt;
(iii) cooling the melt and extruding at temperatures comprising 45-60° C. to facilitate melt formation and/or encapsulation of the calcium salt; and
(iv) drying and pulverising or grinding the extruded mixture into a dry particulate form.

21. The process of claim 20, wherein;
(a) the extruder is operated at a screw speed of about 900 rpm, at a temperature of about 55° C., and/or at a (air) humidity of between about 30% to about 38%;
(b) the dried extruded mixture is pulverized or ground to an average particle size of about 10 to about 30 microns; or
(c) both (a) and (b).

22. (canceled)

23. A food, cosmetic, or personal care product comprising the opacity modifying agent as defined in claim 1.
